(19) United States
(12) Patent Application Publication
(10) Pub. No.: US 2013/0178728 A1
(43) Pub. Date: Jul. 11, 2013

(54) HEART RATE MONITOR HAVING ELECTROSTATIC DISCHARGE PROTECTIVE LAYER

(71) Applicants: Timothy Vandermeiden, Calgary (CA); Christopher J. Kulach, Calgary (CA); Theodorus J. Smit, Calgary (CA); Rogelio A. Rivas, Calgary (CA); Darcy L. Davis, Cochrane (CA)

(72) Inventors: Timothy Vandermeiden, Calgary (CA); Christopher J. Kulach, Calgary (CA); Theodorus J. Smit, Calgary (CA); Rogelio A. Rivas, Calgary (CA); Darcy L. Davis, Cochrane (CA)

(73) Assignee: GARMIN SWITZERLAND GMBH, Schaffhausen (CH)

(21) Appl. No.: 13/526,055

(22) Filed: Jun. 18, 2012

Related U.S. Application Data
(60) Provisional application No. 61/583,248, filed on Jan. 5, 2012.

(51) Int. Cl. A61B 5/0408

(52) U.S. Cl. 600/390

(57) ABSTRACT

A heart rate monitor (HRM) includes an electrostatic discharge protective layer configured to at least partially shield the heart rate monitor from electrostatic discharge (ESD). The heart rate monitor includes a strap configured to be worn on the body of a user that includes signal and ground electrodes. The signal electrode is configured to receive electrical impulses from the heart of the user, while the ground electrode is configured to electrically ground the heart rate monitor to the body of the user. An electrostatic discharge protective layer is disposed on or in the strap over the signal electrode so that the signal electrode is at least substantially positioned between the body of the user and the electrostatic discharge protective layer when the strap is worn. The electrostatic discharge protective layer is electrically coupled to the ground electrode to at least partially shield the signal electrode from electrostatic discharge.

### CROSS-REFERENCE TO RELATED APPLICATIONS

The present application claims the benefit of 35 U.S.C. §119(e) of U.S. Provisional Application Ser. No. 61/583,248, filed Jan. 5, 2012, and titled “ELECTROSTATIC DISCHARGE PROTECTIVE LAYER FOR HEART RATE MONITOR,” which is herein incorporated by reference in its entirety.

### BACKGROUND

When the heart contracts, it generates electrical impulses within the body. Heart rate monitors (HRM) detect these electrical impulses, which are used to measure the heart rate of a user in real time. In many implementations, heart rate monitors wirelessly transmit data describing the detected electrical impulses (heart rate data) to a device such as a runner's watch. The device may then calculate and/or display the user's heart rate to the user and/or record the user's heart rate for later analysis. The device may further use the heart rate data to calculate parameters relating to the user's health or fitness, such as average heart rate over an exercise period, time in a specific heart rate zone, calories burned, breathing rate, heart rate variability, and so forth.

### SUMMARY

A heart rate monitor (HRM) is described that includes an electrostatic discharge protective layer configured to at least partially shield the heart rate monitor from electrostatic discharge (ESD). In one or more implementations, the heart rate monitor includes a strap configured to be worn on the body (e.g., about the torso) of a user. Signal and ground electrodes are disposed at least partially on (e.g., extend along) the inner surface of the strap. The signal electrode is configured to receive electrical impulses from the heart of the user, while the ground electrode is configured to electrically ground the heart rate monitor (HRM) to the body of the user. An electrostatic discharge protective layer is disposed in the strap over the signal electrode so that the signal electrode is at least substantially positioned between the body (e.g., the torso) of the user and the electrostatic discharge protective layer when the strap is worn (e.g., about the torso). The electrostatic discharge protective layer may be electrically coupled to the ground electrode or a connector of the heart rate monitor to at least partially shield the signal electrode from electrostatic discharge. In one or more embodiments, the electrostatic discharge protective layer comprises an extension of the ground electrode over the signal electrode. In other embodiments, the electrostatic discharge protective layer comprises a separate conductive layer that is coupled to the ground electrode so that the ground electrode extends from beneath the conductive layer.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

The detailed description is described with reference to the accompanying figures. The use of the same reference numbers in different instances in the description and the figures may indicate similar or identical items.

FIG. 1 is an illustration of a heart rate monitoring system in an example implementation that includes a heart rate monitor in accordance with the present disclosure.

FIGS. 2A, 2B, and 2C are partial cross-sectional, isometric views of a heart rate monitor, such as the heart rate monitor shown in FIG. 1, further illustrating an example electrostatic discharge protective layer configured to shield the heart rate monitor from electrostatic discharge, wherein the electrostatic discharge protective layer is configured to extend over a heart rate module and a signal electrode.

FIGS. 3A, 3B, and 3C are partial cross-sectional, isometric views of a heart rate monitor, such as the heart rate monitor shown in FIG. 1, further illustrating an example electrostatic discharge protective layer configured to shield the heart rate monitor from electrostatic discharge, wherein the electrostatic discharge protective layer is configured to extend over the signal electrode and under the heart rate module, and wherein the heart rate monitor includes a second electrostatic discharge protective layer configured to extend over the heart rate module.

FIGS. 4A, 4B, and 4C are partial cross-sectional, isometric views of a heart rate monitor, such as the heart rate monitor shown in FIG. 1, further illustrating an example electrostatic discharge protective layer configured to shield the heart rate monitor from electrostatic discharge, wherein the electrostatic discharge protective layer is configured to extend over the signal electrode and under the heart rate module.

FIG. 5 is a partial cross-sectional, isometric views of a heart rate monitor, such as the heart rate monitor shown in FIG. 1, further illustrating an example electrostatic discharge protective layer configured to shield the heart rate monitor from electrostatic discharge, wherein the electrostatic discharge protective layer contacts the body of a user.

FIG. 6 is a partial cross-sectional, isometric views of a heart rate monitor, such as the heart rate monitor shown in FIG. 1, further illustrating an example electrostatic discharge protective layer configured to shield the heart rate monitor from electrostatic discharge, wherein the electrostatic discharge protective layer is configured to extend over the heart rate monitor and electrically couple to a contact of the heart rate monitor.

FIG. 7 is a partial cross-sectional, isometric views of a heart rate monitor, such as the heart rate monitor shown in FIG. 1, further illustrating an example electrostatic discharge protective layer configured to shield the heart rate monitor from electrostatic discharge, wherein the electrostatic discharge protective layer is configured to electrically couple with the ground electrode using a separable connector.

The drawing figures do not limit the heart rate monitoring system, heart rate monitor and/or device to the specific implementations disclosed and described herein. The drawings are not necessarily to scale, emphasis instead being placed upon clearly illustrating elements of the heart rate monitoring system, heart rate monitor and/or device.

### DETAILED DESCRIPTION

### Overview

Many heart rate monitors include a heart rate module coupled to a strap having a fabric construction that is worn on a part of the body of a user such as about the torso (e.g., chest and back), wrist, arm, waist, leg, finger, and so forth. The strap supports two or more electrodes that make direct contact with the skin of the user's chest. One electrode, the signal electrode, receives the electrical impulses generated in the body as the user's heart contracts (e.g., a “heart signal”), while the other electrode, the ground electrode, is configured to ground the heart rate monitor (HRM) to the body of the user (e.g., provides a ground reference potential). In some embodiments, an electrostatic discharge protective layer may also make direct contact with the skin of a user to provide a second ground contact. The electrodes furnish the heart signal and ground to a heart rate module, which interprets the heart signal and transmits data describing the heart rate of a user (i.e., information pertaining to the detected electrical impulses and associated with a user's heart rate, e.g., an EKG signal, a calculated heart rate, etc.) to a paired device via a radio frequency transmitter. The transmitted radio frequency signal can be a simple radio pulse or a unique coded signal (e.g., Bluetooth, ZigBee, ANT, and so forth).

Heart rate monitors are normally worn against the skin of the user's torso so that the electrodes may make direct contact with the skin. However, users will typically wear another garment (e.g., a shirt, blouse, t-shirt, etc.) over the heart rate monitor while exercising. Consequently, as the user moves, the fabric of this garment may rub against the fabric strap and/or heart rate module, causing an electrostatic charge to be built up on and/or within the module and strap. Other sources of electrostatic charge such as skin, hair, accessories, and so forth, may contribute to the buildup of electrostatic charge in the module and strap. This build-up of electrostatic charge may result in electrostatic discharge (ESD), which can induce noise in the heart signal, degrading signal quality. Degraded signal quality can lead to errors in the determined heart rate for the user (e.g., causing the displayed heart rate to be too fast or to slow). Moreover, electrostatic discharge (ESD), if sufficiently great, can interfere with transmission of heart rate data to a device.

Accordingly, a biomedical monitor such as heart rate monitor (HRM) (hereinafter “heart rate monitor”) having an electrostatic discharge protective layer configured to at least partially shield the heart rate monitor from electrostatic discharge (ESD) is described. In one or more implementations, the heart rate monitor includes a strap having a fabric construction that is configured to be worn on (e.g., on or about) the torso of a user. However, in implementations, for example, in other biomedical monitor applications, the strap may be configured to be worn on (e.g., on or about) another part of the body of a user such as a wrist, an arm, the waist, a leg, a finger, and so forth. The strap supports signal and ground electrodes, which are disposed at least partially on (e.g., extend along) the inner surface of the strap. The signal electrode is configured to receive electrical impulses generated in the body of the user as the user's heart beats (e.g., a “heart signal”), while the ground electrode is configured to electrically ground the heart rate monitor (HRM) to the body of the user (e.g., provides a “ground”). However, it is contemplated that in implementations, the signal electrode and the ground electrode may be configured to receive electrical impulses generated in the body of the user that correspond to other bodily functions such as ventilation, breathing rate, skin hydration, and so forth.

An electrostatic discharge protective layer is disposed in the strap over the signal electrode so that the signal electrode is at least substantially positioned between the body (e.g., the torso) of the user and the electrostatic discharge protective layer and the ground electrode at least substantially extends from between the body (e.g., the torso) of the user and the electrostatic discharge protective layer when the strap is worn (e.g., on or about the torso). Thus, the signal electrode is at least substantially covered by the electrostatic discharge protective layer, while the ground electrode remains at least substantially uncovered by the electrostatic discharge protective layer. The electrostatic discharge protective layer may be substantially disposed in the strap or a portion of the electrostatic discharge protective layer may contact the user's skin. For instance, the electrostatic discharge protective layer may extend from the user's skin to an area over the signal electrode so that the signal electrode is at least substantially positioned between the body (e.g., the torso) of the user and the electrostatic discharge protective layer to a point that enables the signal electrode to be substantially covered by the electrostatic discharge protective layer. In embodiments, the electrostatic discharge protective layer may be electrically coupled to ground or mechanically fastened (e.g., sewn in, application of an adhesive, combinations thereof, and so forth) to the ground electrode to at least partially shield the signal electrode from electrostatic discharge. For instance, the electrical coupling may occur through a coupling element, such as a separable connector, through the user's body or through an intermediate element. Ground, as used herein, may be the ground electrode or any signal path that has sufficiently low impedance to earth ground, circuit ground (GND), VCC, a static or varying signal driven by a buffer or any other circuit capable of accepting electrical charge to dissipate electrostatic charge and/or disrupt external electric fields.

The heart rate monitor further includes a heart rate module coupled to the strap. The heart rate module is configured to interpret the heart signal and/or to transmit heart rate data (i.e., information pertaining to the detected electrical impulses and associated with a user's heart rate, e.g., an EKG signal, a calculated heart rate, etc.) to a device. In one or more implementations, a portion of the electrostatic discharge protective layer may extend over the heart rate module so that the heart rate module is at least partially positioned between the torso of the user and the electrostatic discharge protective layer when the strap is worn on the body (e.g., about the torso). In other embodiments, the heart rate monitor may include a second electrostatic discharge protective layer electrically coupled to at least one of the electrostatic discharge protective layer or the ground electrode. The second electrostatic discharge protective layer extends over the heart rate module so that the heart rate module is at least substantially positioned between the second electrostatic discharge protective layer and the electrostatic discharge protective layer. In some embodiments, the electrostatic discharge protective layer may be configured to extend over the signal electrode and under the heart rate module.

The heart rate monitor may further include contacts that electrically couple the heart rate module to the signal electrode, the ground electrode, or the electrostatic discharge protective layer. In one or more implementations, the electrostatic discharge protective layer may extend under the heart rate module so that the electrostatic discharge protective layer is at least substantially positioned between the heart rate module and the inner surface of the strap. In such implementations, the electrostatic discharge protective layer is provided with one or more apertures formed therein through which the contacts extend.

In one or more implementations, the electrostatic discharge protective layer and/or the second electrostatic discharge protective layer may comprise an extension of the ground electrode. In other embodiments, the electrostatic discharge protective layer comprises a separate conductive layer coupled to the ground electrode so that the ground electrode extends from above, beneath, or along a side of the conductive layer. A housing (sheath) may at least substantially enclose the heart rate module as well as the portion of the electrostatic discharge protective layer extending over the heart rate module or the second electrostatic discharge protective layer.

In one or more implementations, the electrostatic discharge protective layer may make direct contact with the skin of the user, such as the user's chest. The electrostatic discharge protective layer may extend over the signal electrode and over the heart rate monitor. In implementations, the electrostatic discharge protective layer may substantially cover the signal electrode without being electrically coupled with any element in the strap. In other implementations, the electrostatic discharge protective layer may substantially cover the signal electrode and electrically couple with the ground electrode, a connector of the heart rate monitor, or other conductive element in the strap.

In one or more implementations, the electrostatic discharge protective layer may be electrically coupled with a connector of the heart rate monitor. For instance, the electrostatic discharge protective layer may substantially cover the signal electrode and electrically couple with the connector of the heart rate monitor by extending under the heart rate monitor so that the electrostatic discharge protective layer is at least substantially positioned between the heart rate monitor and the inner surface of the strap or by extending over the heart rate monitor so that the heart rate monitor is at least partially positioned between the torso of the user and the electrostatic discharge protective layer when the strap is worn on the body (e.g., about the torso).

It is to be understood that the shielding techniques described herein may be implemented in many configurations. For instance, the electrostatic discharge protective layer may be disposed over the ground electrode instead of the signal electrode. In embodiments, the signal electrode may be substantially uncovered by the electrostatic discharge protective layer while the ground electrode is substantially covered. Such embodiments may have an advantage by reducing manufacturing costs. In some embodiments, an electrostatic discharge protective layer may be disposed over the ground electrode so that the ground electrode is at least substantially positioned between the body of the user and the electrostatic discharge protective layer. In one or more implementations, a portion of the electrostatic discharge protective layer may extend from the ground electrode over the heart rate module so that the heart rate module is at least partially positioned between the user's body and the electrostatic discharge protective layer when the strap is worn on the body. In other implementations, a portion of the electrostatic discharge protective layer may extend from the ground electrode and beneath (i.e., under) the heart rate module. A portion of the electrostatic discharge protective layer may also extend over the ends and sides of the heart rate module to further shield the heart rate module. In implementations, the electrostatic discharge protective layer may be electrically coupled to the heart rate monitor, ground electrode, any element within the strap or a portion of the strap using a separable connector, or indirectly through a connector that electrically couples the ground electrode to the heart rate module. In other implementations, the electrostatic discharge protective layer may be mechanically fastened (e.g., sewn in, application of an adhesive, combinations thereof, and so forth) to the heart rate monitor, ground electrode, any element within the strap or a portion of the strap using a separable connector.

In some embodiments, both the ground and signal electrodes may be shielded using one or more electrostatic discharge protective layers. In some configurations, one or more electrostatic discharge protective layers are electrically coupled directly or indirectly with a signal other than earth ground. For instance, an electrostatic discharge protective layer may be electrically coupled with a voltage, such as VCC, a static or varying signal driven by a buffer or any other electrical circuit capable of accepting electrical charge to dissipate electrostatic charge and/or disrupt external electric fields. In embodiments, a plurality of electrostatic discharge protective layers may be electrically coupled with distinct voltages. For instance, a first electrostatic discharge protective layer may be disposed over a ground electrode and electrically coupled with a first voltage and a second electrostatic discharge protective layer may be disposed over a signal electrode and electrically coupled with a second voltage. Many other configurations are contemplated.

### Example Environment

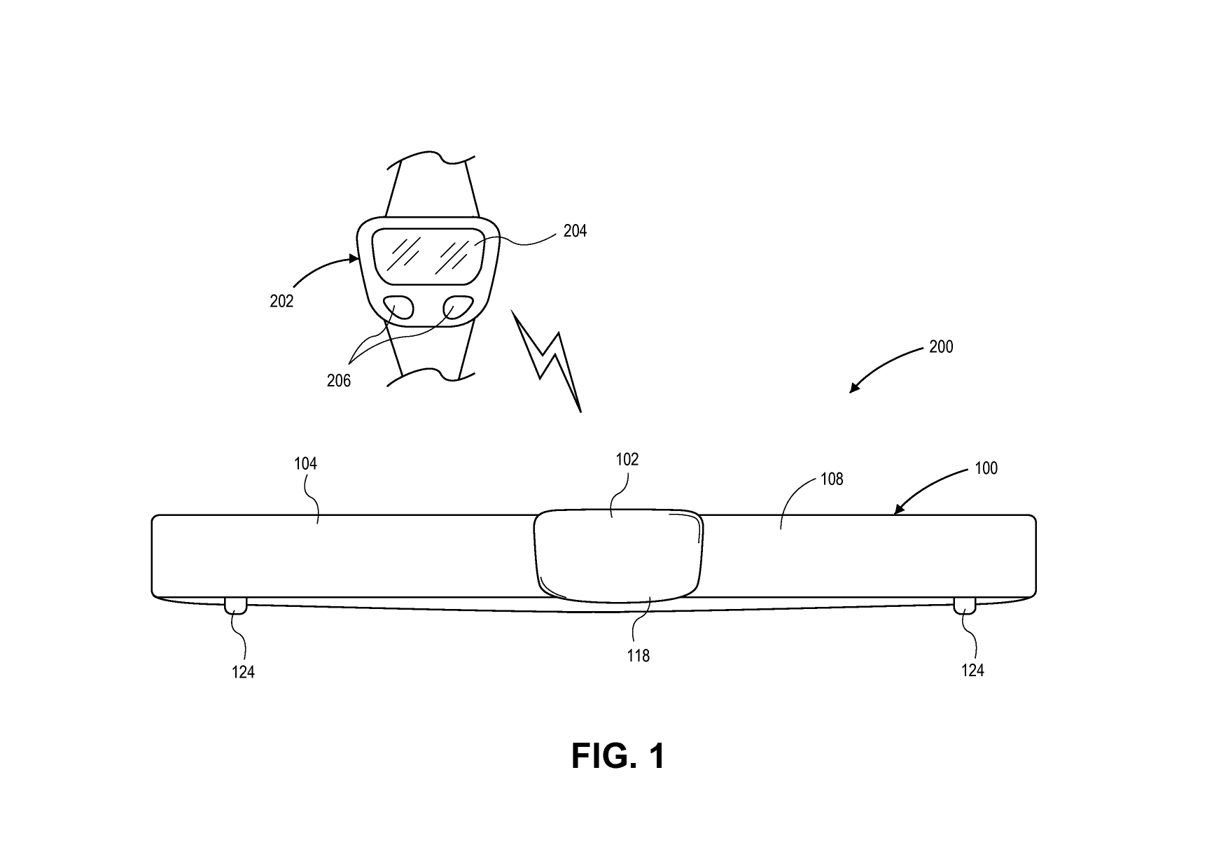

FIG. 1 illustrates a heart rate monitor 100 in accordance with an example implementation of the present disclosure. The heart rate monitor 100 is configured to be worn by a user to detect electrical impulses from the user's body that can be used to measure, for instance, the heart rate of a user in real time. As shown, the heart rate monitors 100 include a heart rate module 102 coupled to a supporting structure for supporting (holding) the heart rate monitor 100 proximate to the body (e.g., the chest) of a user. In the illustrated implementations, the supporting structure comprises a strap 104 that may be worn about the torso (e.g., chest and back) of a user. However, it is contemplated that in other implementations, the supporting structure may also comprise a garment worn by the user such as a sports bra, a fitted shirt, a compression shirt, and so forth. In such implementations, the strap 104 may comprise a component or portion of such a garment that is configured to hold the heart rate module 102. Moreover, it is contemplated that the strap 104 may be configured to be worn on another part of the body of a user such as about the user's wrist, arm, waist, leg, finger, and so forth, for example, in other biomedical monitor implementations.

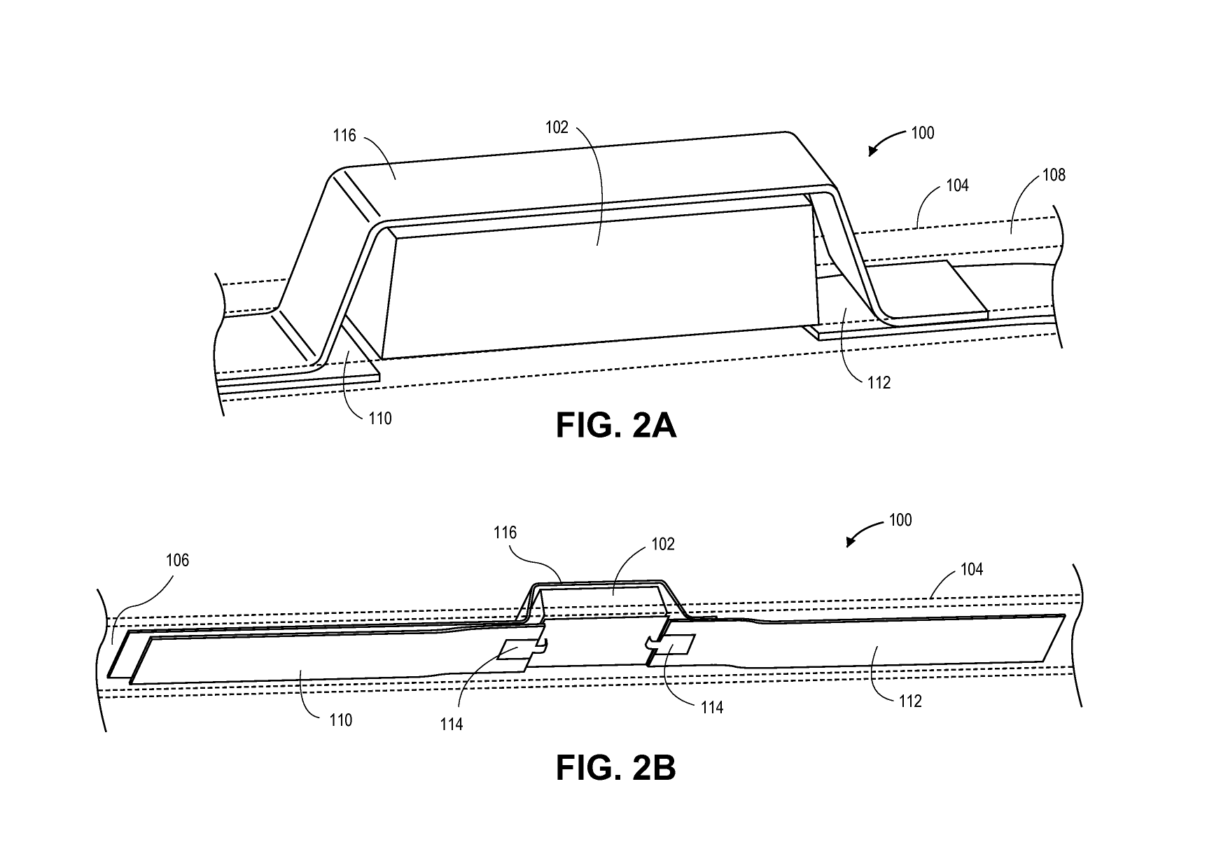

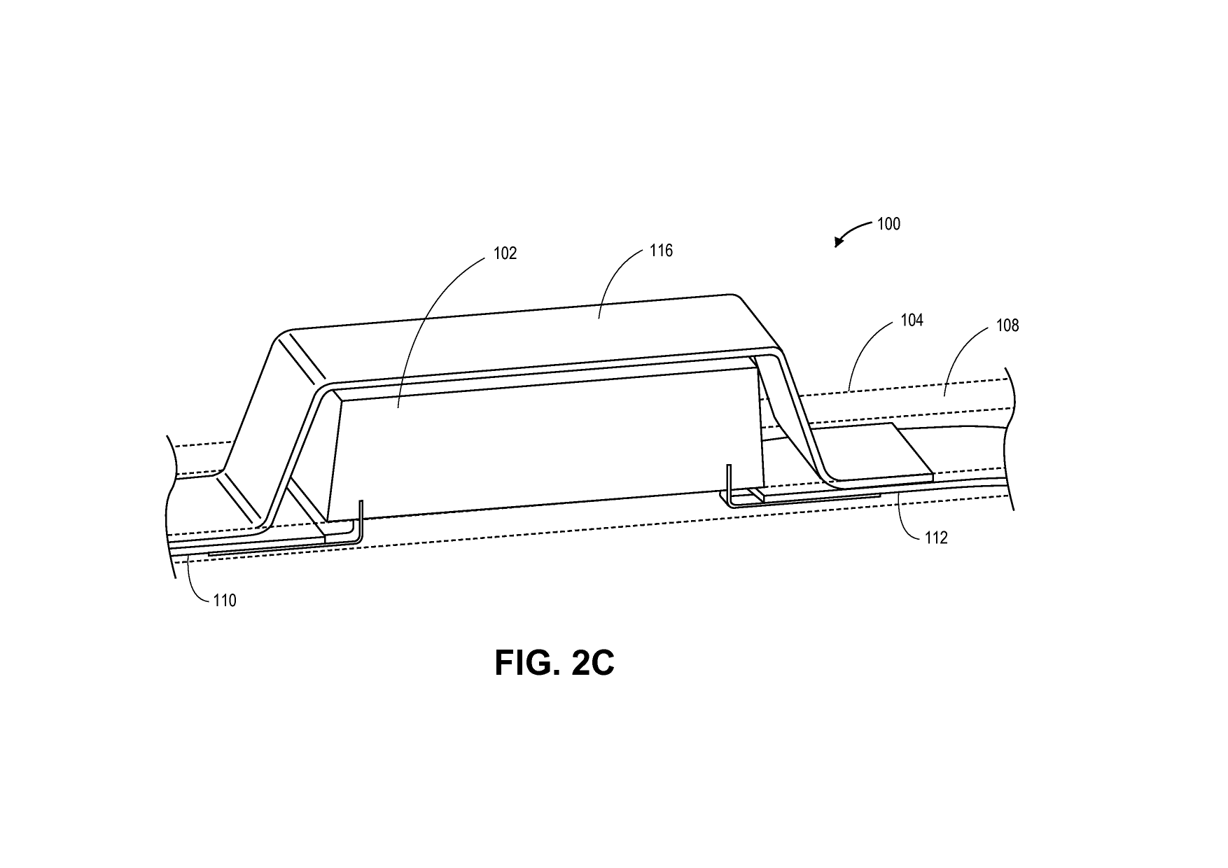

As shown in FIGS. 2A through 7, the strap 104 includes an inner surface 106 (the surface of side of the strap 104 adjacent to the user's body when the user wears the heart rate monitor 100) and an outer surface 108 (the surface of the side of the strap 104 opposite the user's body when the user wears the heart rate monitor 100). The strap 104 may include two or more electrodes. For example, in the illustrated implementations, the strap 104 includes a signal electrode 110 and a ground electrode 112. The signal electrode 110 is configured to receive (detect) electrical impulses from the heart of a user generated within the user's body when the heart contracts (e.g., the “heart signal”). The ground electrode 112 is positioned in proximity to the signal electrode 110 for connecting the heart rate monitor 100 to an electrical ground (e.g., via the user's body). In some implementations, the signal electrode 110 and the ground electrode 112 may be configured to receive (detect) electrical impulses generated in the body of the user that correspond to other bodily functions such as ventilation, breathing rate, skin hydration, and so forth in addition to, or instead of, electrical impulse from the heart. As shown, the signal and ground electrodes 110 and 112 are positioned on (e.g., the signal and ground electrodes 110 and 112 longitudinally extend along) the inner surface 106 of the strap 104 and are configured to detect electrical impulses from a user's body against which the strap 104 is worn However, it is contemplated that the signal and ground electrodes 110 and 112 may also be at least partially positioned within the strap 104 (e.g., a covering layer may be provided over at least part of the signal and/or ground electrodes 110 and 112).

The strap 104 may be fabricated from one or more layers of suitable materials such as flexible plastic, soft fabric, and so forth, configured to conform to the user's body and place the electrodes 110 and 112 in direct contact with the skin of the user's chest. Thus, the strap 104 may have an outer covering layer fabricated of fabric, flexible plastic, and so forth that forms the outer surface 108 and an inner covering layer fabricated of fabric, flexible plastic, and so forth that forms the inner surface 106. For clarity of illustration, these covering layers are shown using hidden lines in FIGS. 2A through 7. Further, the strap 104 may include adjustment hardware, such as clasps 124 (FIG. 1), hook-and-loop fastener material, and so forth, to allow the user to tighten the strap 104 against his or her body (e.g., against his or her torso).

Materials used to manufacture the strap 104 (and heart rate monitor 100) may be selected to reduce the amount of electrical charge/static generated during use. For example, different materials having different tendencies for attracting or repelling free electrical charge may be selectively used cooperatively in the manufacture of the strap 104 to limit the buildup of electrostatic charge, reducing the noise in the heart rate monitor system 200.

The strap 104 and/or heart rate monitor 100 may rub against a garment or accessory worn by the user and generate electrostatic charges as a user moves. In some embodiments, the materials used to manufacture the strap 104 and/or heart rate monitor 100 may be selected to work in conjunction with a garment or accessory worn by the user that may work together to reduce the build-up of electrical charge/static generated during use. It is contemplated that a garment worn by the user may include a sports bra, a fitted shirt, a compression shirt, or any materials worn on the torso of the user or another part of the body of a user, such as about the user's wrist, arm, waist, leg, finger, and an accessory may include an additional belt or strap.

The signal and ground electrodes 110 and 112 may be attached to the strap 104 (e.g., to the inner covering layer of the strap 104) via a suitable attachment technique such as sewing, use of an adhesive, and so forth. The signal and ground electrodes 110 and 112 are connected (e.g., electrically coupled) to the heart rate module 102, and provide the heart signal and ground to the heart rate module 102. The heart rate module 102 may be configured to interpret and/or transmit the heart signal to a device (e.g., the device 202 described with reference to FIG. 1). The signal and ground electrodes 110 and 112 can be directly connected to the heart rate module 102 and/or connected to the heart rate module 102 using, for example, one or more contacts 114 (which may include a signal contact coupled to the signal electrode 110 and a ground contact coupled to the ground electrode 112). The contacts 114 may, in some implementations, permit removal of the heart rate module 102 from the strap 104 (e.g., permit disconnection and/or reconnection of the heart rate module from and/or to the signal and ground electrodes 110 and 112).

The heart rate monitor 100 includes an electrostatic discharge protective layer 116 configured to at least partially shield the signal electrode 110 and/or the heart rate module 102 from electrostatic discharge (ESD). The electrostatic discharge protective layer 116 can include one or more sheets (layers) constructed from, but not necessarily limited to, a conductive material, such as a metal foil, a metalized fabric, a metal mesh, and so forth. In implementations, the electrostatic discharge protective layer 116 may be a separate layer inserted between and/or attached to (e.g., via sewing, adhesive, etc.) one or more of the layers of material from which the strap 104 is fabricated. However, in other implementations, the electrostatic discharge protective layer 116 may itself comprise one or more of the layers of material (e.g., the outer covering layer, an intermediate layer, the inner covering layer, etc.) from which the strap 104 is fabricated, or a portion thereof. In implementations where the electrostatic discharge protective layer 116 is formed on or as part of the outer covering layer of the strap 104, it is contemplated that the outer surface of the electrostatic discharge protective layer 116 may form part of the outer surface of the 108 of the strap 104. In implementations where the electrostatic discharge protective layer 116 is formed on or as part of the inner covering layer of the strap 104, it is contemplated that the inner surface of the electrostatic discharge protective layer 116 may form part of the inner surface of the 106 of the strap 104.

In embodiments, the electrostatic discharge protective layer 116 is electrically coupled to the ground electrode 112 to discharge electrostatic charge within the strap 104 to electrical ground and/or to disrupt the electric field established by the electrostatic charge. In this manner, electrostatic discharge (ESD) shielding can be furnished to the heart rate monitor 100 (e.g., the signal electrode 110 and heart rate module 102) without the addition of dedicated electrodes for dissipating electrostatic charge to the body of the user. In other embodiments, the electrostatic discharge protective layer 116 is electrically coupled to any signal path that has sufficiently low impedance to earth ground, circuit ground (GND), VCC, a static or varying signal driven by a buffer or any other circuit capable of accepting electrical charge to dissipate electrostatic charge and/or disrupt external electric fields.

The electrostatic discharge protective layer 116 may be disposed in the strap 104 over the signal electrode 110 so that the signal electrode 110 is at least substantially positioned between the body (e.g., the torso) of the user and the electrostatic discharge protective layer 116 and the ground electrode 112 at least substantially extends from between the body (e.g., the torso) of the user and the electrostatic discharge protective layer 116 when the strap 104 is worn (e.g., about the torso of the user). Thus, as shown in FIGS. 2A through 7, the signal electrode 110 is at least substantially covered by the electrostatic discharge protective layer 116, while the ground electrode 112 remains at least substantially uncovered by the electrostatic discharge protective layer 116. In this manner, the size of the electrostatic discharge protective layer 116 may be reduced. Consequently, less of the material (e.g., metal foil, metalized fabric, metal mesh, and so forth) from which the electrostatic discharge protective layer 116 is fabricated is required to manufacture the strap 104. Thus, a heart rate monitor 100 (or other biomedical monitor) may be produced that has a strap 104 furnishing electrostatic discharge protection, which has reduced weight, less bulk (e.g., less thickness), and lower cost than would be possible if both electrodes 110, 112 were shielded.

In implementations, the electrostatic discharge protective layer 116 can be an extension of the ground electrode 112 (e.g., the ground electrode 112 is extended over the signal electrode 110 and/or the heart rate module 102). In such implementations, the electrostatic discharge protective layer 116 and the ground electrode 112 may be fabricated from the same material (e.g., a metal foil, a metalized fabric, a metal mesh, etc.). Thus, the material may be viewed as having a portion that comprises (e.g., functions as) the electrostatic discharge protective layer 116 and a portion that comprises (e.g., functions as) the ground electrode 112. It will be appreciated that the portion of material functioning as the electrostatic discharge protective layer 116 need not be (is not) folded over the portion that functions as the ground electrode 112 to cover that portion.

In other implementations, the electrostatic discharge protective layer 116 may comprise a separate conductive layer that is electrically coupled to the ground electrode 112 via a suitable electromechanical fastening technique such as sewing, soldering, application of a conductive adhesive, wiring, combinations thereof, and so forth. In such implementations, the electrostatic discharge protective layer 116 and ground electrode 112 may be fabricated of different materials. In this manner, the electrostatic discharge protective layer 116 can be fabricated of a material that has properties selected to furnish optimized protection against electrostatic discharge (ESD). For example, the electrostatic discharge protective layer 116 may be fabricated of a material that possesses high electrical resistance to limit electrical interaction between the electrostatic discharge protective layer 116 and the signal electrode 110. Such materials (materials having high electrical resistance) may still provide a statically conductive path for static discharge to pass along the material to the ground electrode 112 and be dissipated to the body of the user. As noted, the ground electrode 112 may at least substantially extend from between the body (e.g., the torso) of the user and the electrostatic discharge protective layer 116 when the strap 104 is worn on the body (e.g., about the torso) so that the ground electrode 112 remains at least substantially uncovered by the electrostatic discharge protective layer 116. For example, a portion of the electrostatic discharge protective layer 116 may overlap the ground electrode 112 so that the electrostatic discharge protective layer 116 extends over (or under) the ground electrode 112 by an amount that is limited in size (e.g., area) to a size (area) that is sufficient to allow attachment of the electrostatic discharge protective layer 116 to the ground electrode 112, but which does not otherwise appreciably extend over (or under) the ground electrode 112. Thus, the electrostatic discharge protective layer 116 may extend over (or under) the ground electrode by an amount that allows a seam to be formed in the respective materials of the electrostatic discharge protective layer 116 and the ground electrode 112, by an amount that allows the respective materials of the electrostatic discharge protective layer 116 and ground electrode 112 to be adhered or fastened (e.g., with fasteners such as rivets, snaps, staples, etc.) together, and so forth. It is contemplated that the size of the overlap may vary depending on the materials selected for fabrication of the electrostatic discharge protective layer 116 and the ground electrode 112, sewing/fastening/adhesion techniques used during manufacture, and so forth.

The electrostatic discharge protective layer 116 may be positioned (e.g., as a layer within or on the surface of the strap 104) so that it generally extends over the signal electrode 110. For example, in the implementation shown, the electrostatic discharge protective layer 116 may be positioned at least substantially between the signal electrode 110 and the outer surface 108 of the strap 104 so that the electrostatic discharge protective layer 116 is at least substantially positioned above the signal electrode 110 in relation to the body (e.g., the torso) of the user (e.g., the signal electrode 110 is positioned between the torso of a user and the electrostatic discharge protective layer 116) when the strap 104 is worn on the body (e.g., about the torso) of the user. Thus, when viewed from the outer surface 108 of the strap 104, the electrostatic discharge protective layer 116 may at least substantially cover, and in implementations, may fully cover (e.g., extend beyond) the signal electrode 110 (and, in some instances, the heart rate module 102). For example, the electrostatic discharge protective layer 116 may fully enclose the signal electrode 110 and/or heart rate module 102 (i.e., shielding from electrostatic discharge (ESD) above, below, and along all four sides). As noted, the electrostatic discharge protective layer 116 does not extend substantially over the ground electrode 112. As shown in FIGS. 2A through 2C, the electrostatic discharge protective layer 116 may further extend over the heart rate module 102 (in addition to the signal electrode 110) so that the heart rate module 102 is at least substantially positioned between the body (e.g., the torso) of a user and the electrostatic discharge protective layer 116 when the strap 104 is worn on a part of the user's body (e.g., about the torso of the user). For example, in the implementation shown, a portion of the electrostatic discharge protective layer 116 may exit the outer surface 108 of the strap 104 and pass over the top (the side opposite the user's torso) of the heart rate module 102 to shield the heart rate module 102. The electrostatic discharge protective layer 116 may then enter back into the outer surface 108 of the strap 104 and extend to cover the signal electrode 110 or a substantial portion thereof. The portion of the electrostatic discharge protective layer 116 may also extend over the ends and sides of the heart rate module 102 to further shield the heart rate module 102. In other implementations, the electrostatic discharge protective layer 116 may be electrically coupled to the ground electrode 112 using a separable connector, or indirectly through a connector that electrically couples the ground electrode 112 to the heart rate module 102. Thus, the portion of the electrostatic discharge protective layer 116 may be electrically connected to the ground electrode 112 permanently or separably in any suitable way. In embodiments, a portion of the electrostatic discharge protective layer 116 may pass over the heart rate module 102. As a result, the portion of the electrostatic discharge protective layer 116 that passes over the heart rate module 102 may be electrically connected to the ground electrode 112 permanently or separably in any suitable way. It is to be understood that electrical coupling may be direct or indirect. For instance, electrical coupling may occur through a coupling element, such as a separable connector, through the user's body or through an intermediate element.

In implementations, it is contemplated that the electrostatic discharge protective layer 116 may be electrically coupled to the user's body itself (e.g., instead of being electrically coupled to the ground electrode 112), either passively or using a third electrode. In this manner, the amount of noise entering the heart rate monitor system 200 at the electrical reference location (e.g., through the signal electrode 110 or heart rate module 102) can be reduced. Connection of the electrostatic discharge protective layer 116 to the body can be made using a variety of materials with differing levels of conductivity to provide connection and/or dissipation of the electrostatic charge.

The electrostatic discharge protective layer 116 thus provides a grounded electrical net configured to act as a protective layer against electrostatic discharge entering (e.g., impacting the performance of) the heart rate module 102, its contacts 114 and/or the signal electrode 110. As shown in FIG. 1, the heart rate module 102 and the portion of the electrostatic discharge protective layer 116 extending over the heart rate module 102 may be covered (i.e., enclosed) and/or concealed within a housing 118 (e.g., a hard plastic housing, a soft plastic or fabric sheath or covering, etc.) to protect against wear, environmental damage, and so forth.

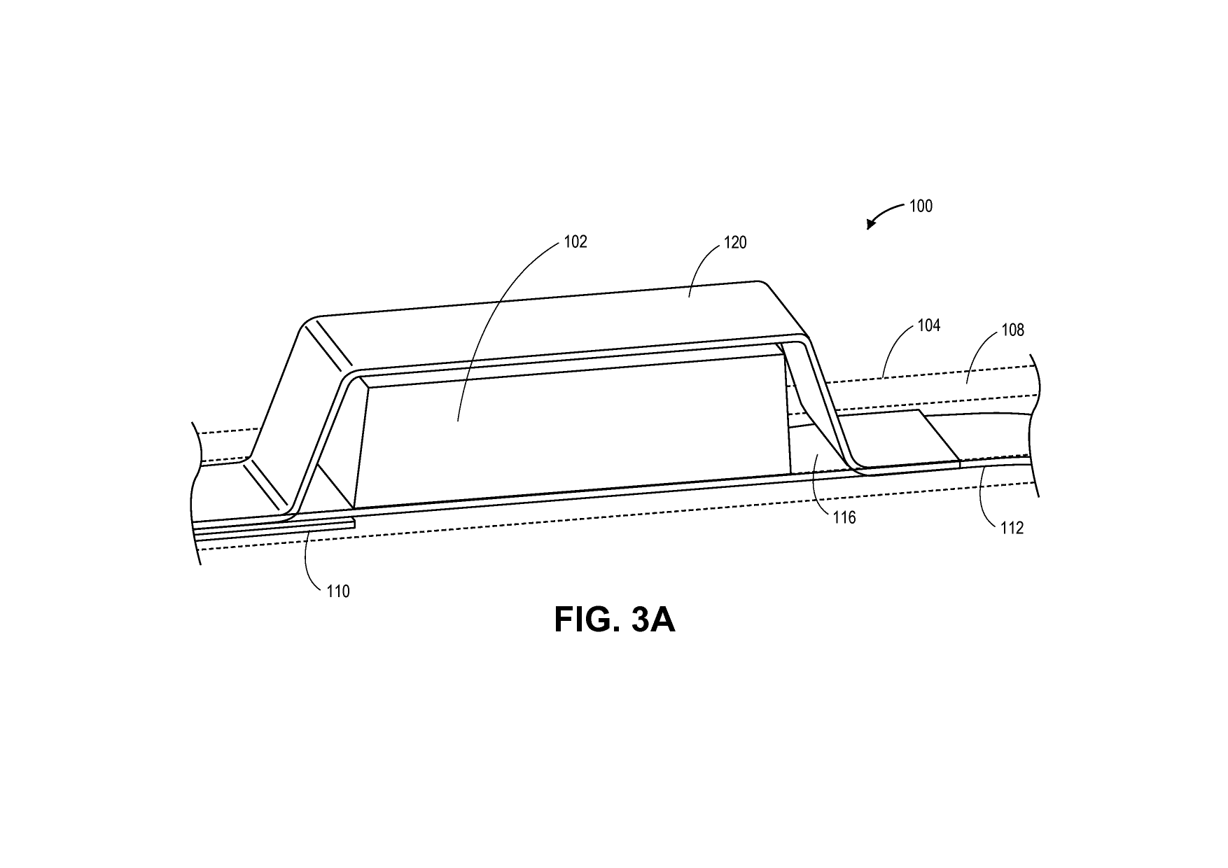

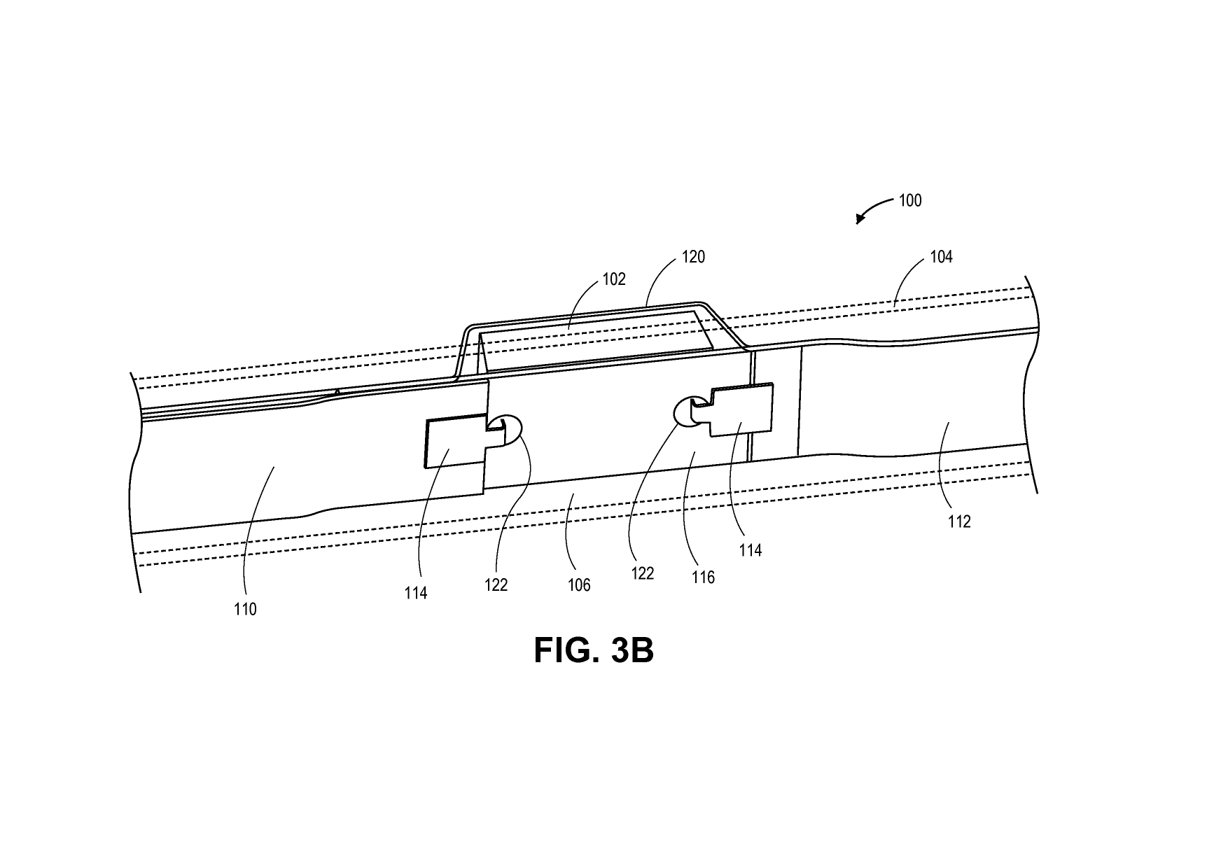

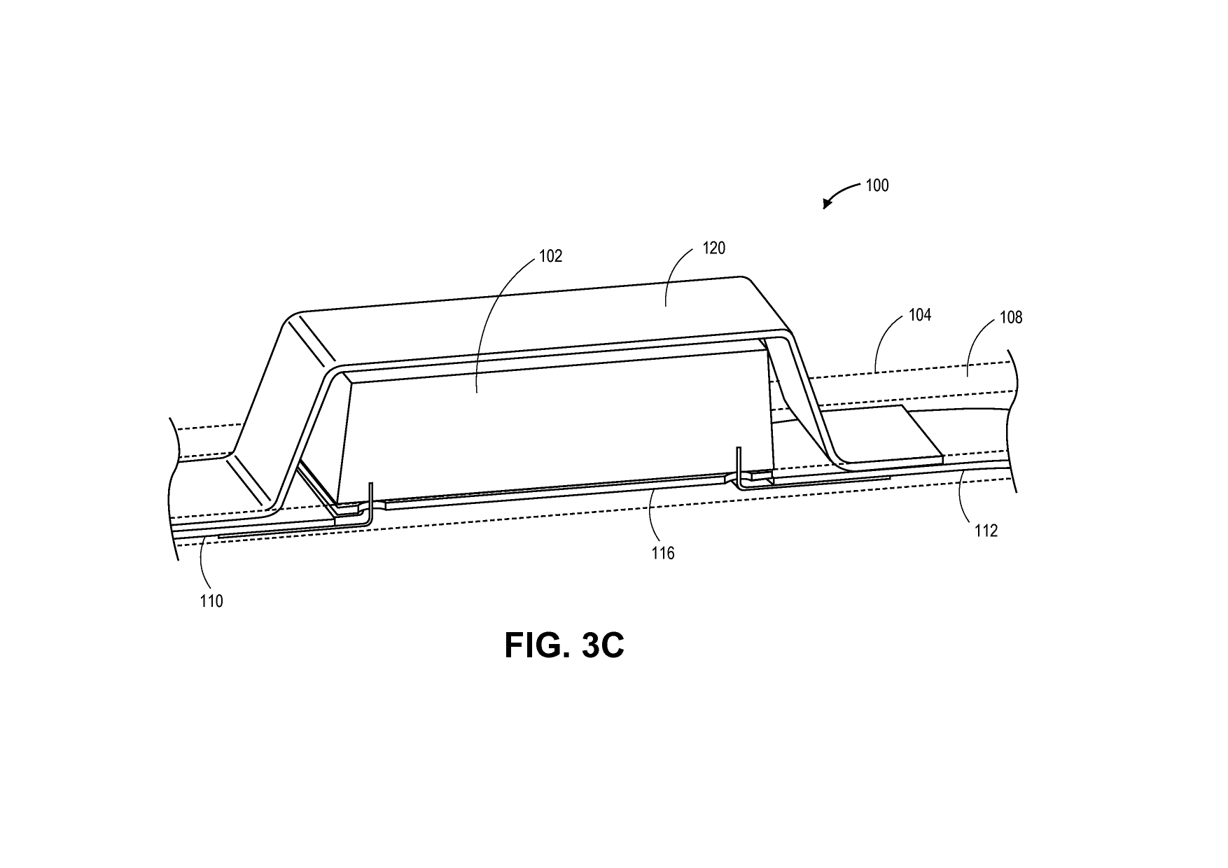

As illustrated in FIGS. 3A through 3C, the electrostatic discharge protective layer 116 may also extend under the heart rate module 102 (and over the signal electrode 110). In some configurations, the electrostatic discharge protective layer 116 may shield the contacts 114 from electrostatic discharge. As shown in FIG. 3B, the electrostatic discharge protective layer 116 may be provided with one or more apertures 122 formed therein, through which the contacts 114 extend to electrically couple the signal and ground electrodes 110 and 112 to the heart rate module 102.

The heart rate monitor 100 may include a second electrostatic discharge protective layer 120 configured to extend over the heart rate module 102 so that the heart rate module 102 is at least substantially positioned between the second electrostatic discharge protective layer 120 and the electrostatic discharge protective layer 116. For example, as shown in FIGS. 3A through 3C, the second electrostatic discharge protective layer 120 may exit the outer surface 108 of the strap 104 and pass over the top (side opposite the user's torso) of the heart rate module 102. The material of the second electrostatic discharge protective layer 120 may then enter back into the outer surface 108 of the strap 104. The second electrostatic discharge protective layer 120 may also extend over the ends and sides of the heart rate module 102 to further shield the module 102.

The second electrostatic discharge protective layer 120 is configured to at least partially shield the heart rate module 102 from electrostatic discharge (ESD). The second electrostatic discharge protective layer 120 can be constructed from, but is not necessarily limited to, a conductive material, such as a metal foil, a metalized fabric, a metal mesh, and so forth. In implementations, the second electrostatic discharge protective layer 120 may be a separate layer having at least a portion of its perimeter edges (e.g., ends) inserted between and/or attached to (e.g., via sewing, adhesive, etc.) one or more of the layers of material from which the strap 104 is fabricated. However, in other implementations, the second electrostatic discharge protective layer 120 may itself comprise one or more of the layers of material (e.g., the outer covering layer, an intermediate layer, etc.) from which the strap 104 is fabricated, or a portion thereof that is extended to cover the heart rate module 102.

The second electrostatic discharge protective layer 120 is electrically coupled to the ground electrode 112 and/or the electrostatic discharge protective layer 116 to discharge electrostatic charge within the strap 104 to electrical ground and/or to disrupt the electric field established by the electrostatic charge. In implementations, the second electrostatic discharge protective layer 120 can be an extension of the ground electrode 112 and/or the electrostatic discharge protective layer 116. In some configurations, the ground electrode 112 or the electrostatic discharge protective layer 116 is extended over the heart rate module 102 by extension of the second electrostatic discharge protective layer 120. In other configurations, the electrostatic discharge protective layer 116 is extended both over and under the heart rate module 102 by extension of the second electrostatic discharge protective layer 120. Other configurations are contemplated. In such implementations, the second electrostatic discharge protective layer 120, electrostatic discharge protective layer 116 and/or the ground electrode 112 may be fabricated from the same material (e.g., a metal foil, a metalized fabric, a metal mesh, etc.). Thus, the material may be viewed as having a portion that comprises (e.g., functions as) the second electrostatic discharge protective layer 120, a portion that comprises (e.g., functions as) the electrostatic discharge protective layer 116, and/or a portion that comprises (e.g., functions as) the ground electrode 112. It will be appreciated that the portions of material functioning as the second electrostatic discharge protective layer 120 and/or the electrostatic discharge protective layer 116 need not be (are not) folded over the portion that functions as the ground electrode 112 to cover that portion.

In other implementations, the second electrostatic discharge protective layer 120 may comprise a separate (additional) conductive layer that is electrically coupled to the ground electrode 112 and/or the electrostatic discharge protective layer 116 via a suitable electromechanical fastening technique such as sewing, soldering, application of a conductive adhesive, wiring, combinations thereof, and so forth. In such implementations, the electrostatic discharge protective layer 116, the second electrostatic discharge protective layer 120 and/or the ground electrode 112 may be fabricated of different materials. In this manner, the electrostatic discharge protective layer 116 and/or the second electrostatic discharge protective layer 120 can be fabricated of a material that has properties selected to furnish optimized protection against electrostatic discharge (ESD) of the components shielded. For example, the electrostatic discharge protective layer 116 and/or the second electrostatic discharge protective layer 120 may be fabricated of materials (e.g., a common material or two or more different materials) that possess high electrical resistance to limit electrical interaction between the electrostatic discharge protective layer 116 and the signal electrode 110 and/or the second electrostatic discharge protective layer 120 and the heart rate module 102. As noted, such materials (materials having high electrical resistance) may still provide a statically conductive path for static discharge to pass along the material to the ground electrode 112 and be dissipated to the body of the user.

The ground electrode 112 may at least substantially extend from between the torso of the user and the second electrostatic discharge protective layer 120 (and electrostatic discharge protective layer 116) when the strap 104 is worn on the body (e.g., about the torso) so that the ground electrode 112 remains at least substantially uncovered by the second electrostatic discharge protective layer 120 and/or the electrostatic protective discharge layer 116. Where the second electrostatic discharge protective layer 120 is attached to the ground electrode 112, it is contemplated that a portion of the second electrostatic discharge protective layer 20 may overlap the ground electrode 112 so that the second electrostatic discharge protective layer 116 extends over (or under) the ground electrode 112 by an amount that is limited in size (e.g., area) to a size (area) that is sufficient to allow attachment of the second electrostatic discharge protective layer 120 to the ground electrode 112, but which does not otherwise appreciably extend over (or under) the ground electrode 112. Thus, the second electrostatic discharge protective layer 116 may extend over (or under) the ground electrode by an amount that allows a seam to be formed in the respective materials of the second electrostatic discharge protective layer 120, the electrostatic discharge protective layer 116 and/or the ground electrode 112, by an amount that allows the respective materials of the second electrostatic discharge protective layer 120, the electrostatic discharge protective layer 116 and/or ground electrode 112 to be adhered or fastened (e.g., with fasteners such as rivets, snaps, staples, etc.) together, and so forth. It is contemplated that the size of the overlap may vary depending on the materials selected for fabrication of the second electrostatic discharge protective layer 120, the electrostatic discharge protective layer 116, and/or the ground electrode 112, sewing/fastening/adhesion techniques used during manufacture, and so forth.

When viewed from the outer surface 108 of the strap 104, the second electrostatic discharge protective layer 120 may at least substantially cover, and in implementations, may fully cover the heart rate module 102. For example, the second electrostatic discharge protective layer 120 may fully enclose the heart rate module 102 (i.e., shielding from electrostatic discharge (ESD) above, below, and along all four sides of the heart rate module 102). In the implementations illustrated, the second electrostatic discharge protective layer 120 does not extend substantially over the ground electrode 112.

The heart rate module 102 and at least the portion of the second electrostatic discharge protective layer 120 extending over the heart rate module 102 may be covered and/or concealed within a housing 118 (e.g., a hard plastic housing, a soft plastic or fabric sheath or covering, etc.), as shown in FIG. 1, to protect against wear, environmental damage, and so forth.

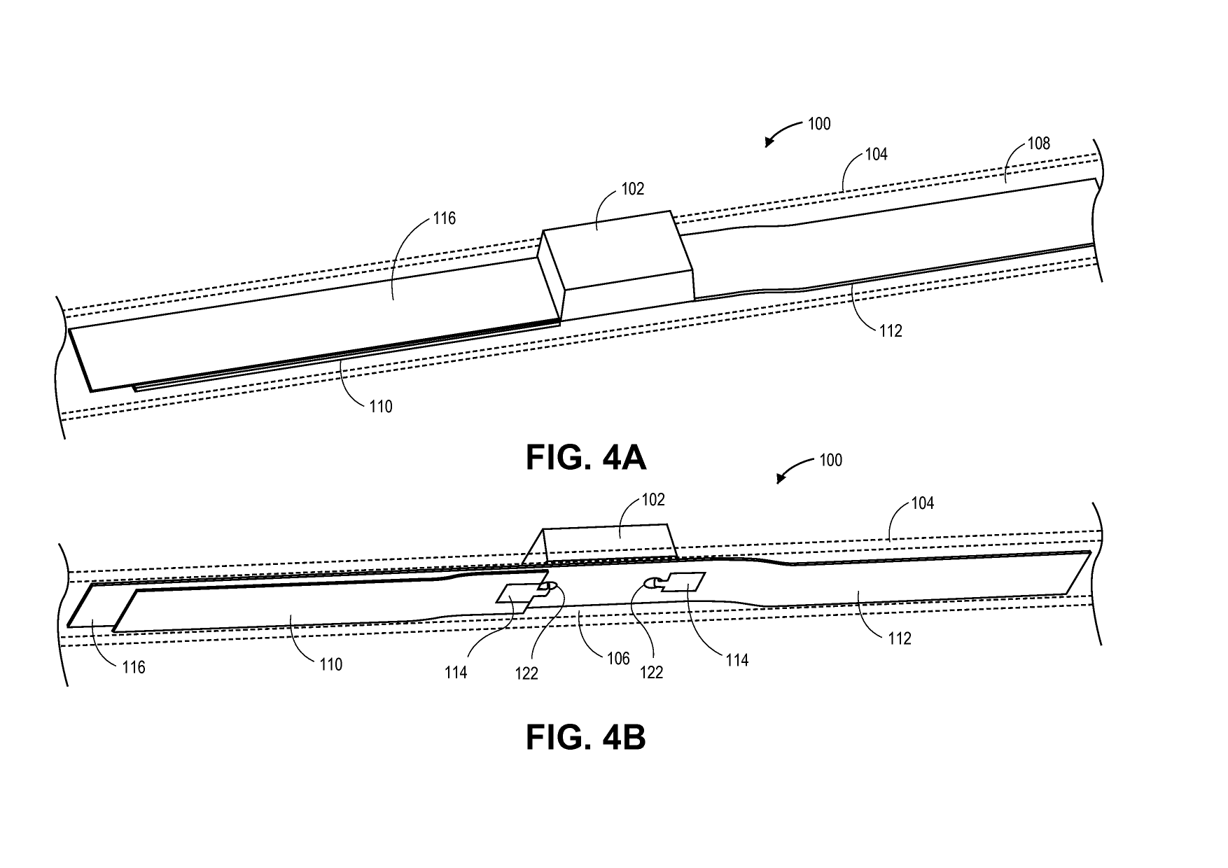

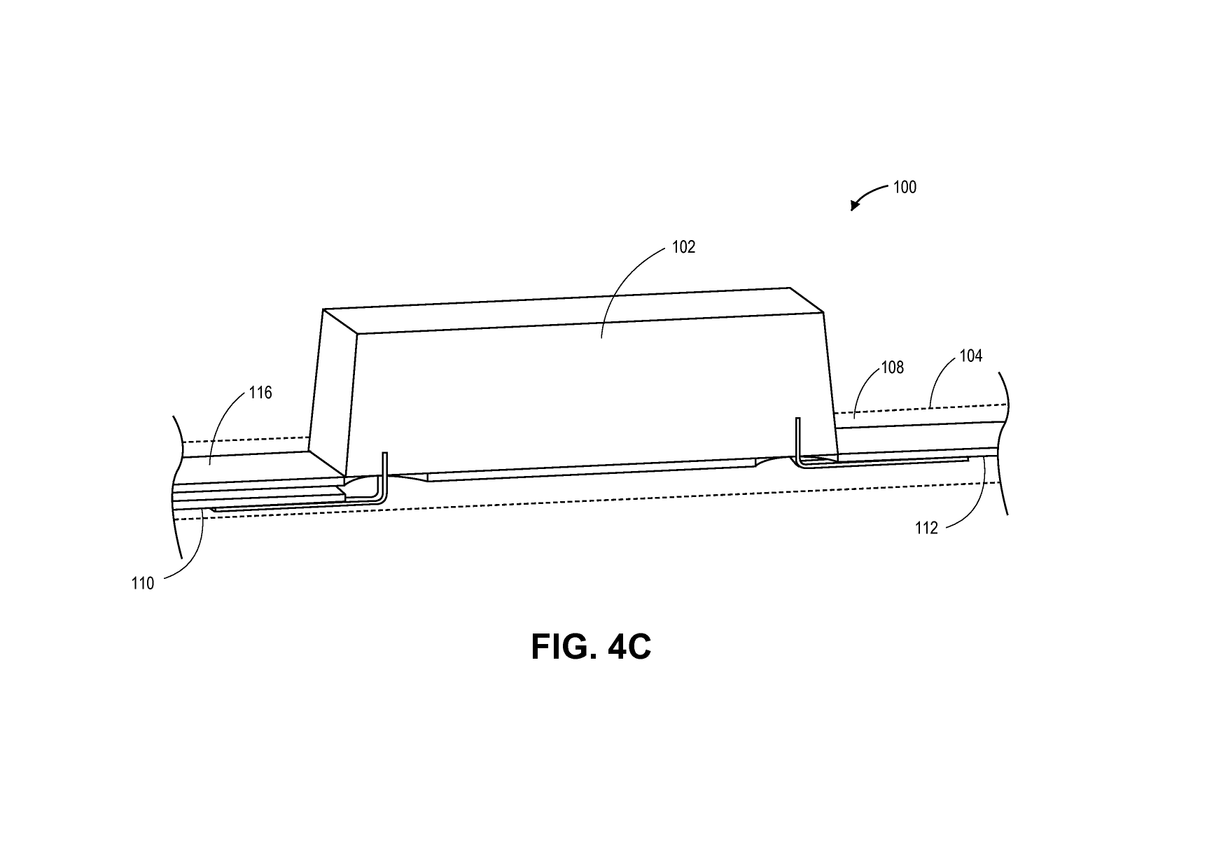

As illustrated in FIGS. 4A through 4C, in some embodiments, the heart rate module 102 is not separately shielded from electrostatic discharge (ESD). Instead, the electrostatic discharge protective layer 116 may extend under the heart rate module 102 and over the signal electrode 110. The electrostatic discharge protective layer 116 may be provided with one or more apertures 122 formed therein, through which the contacts 114 extend to electrically couple the signal and ground electrodes 110 and 112 to the heart rate module 102. It is contemplated that, in such implementations, the heart rate module 102 may be shielded from electrostatic discharge (ESD) internally, or it may be unshielded from above and along all four sides. Further, in such implementations, the heart rate module 102 may be covered and/or concealed within a housing 118 (e.g., a hard plastic housing, a soft plastic or fabric sheath or covering, etc.), as shown in FIG. 1, which may furnish shielding from electrostatic discharge (ESD). For example, the housing 118 may be fabricated of (e.g., fabricated entirely of, or include layers or inserts fabricated of) a conductive material such as a metal, metalized plastic or composite, metal mesh, and so forth, to shield the heart rate module from electrostatic discharge (ESD).

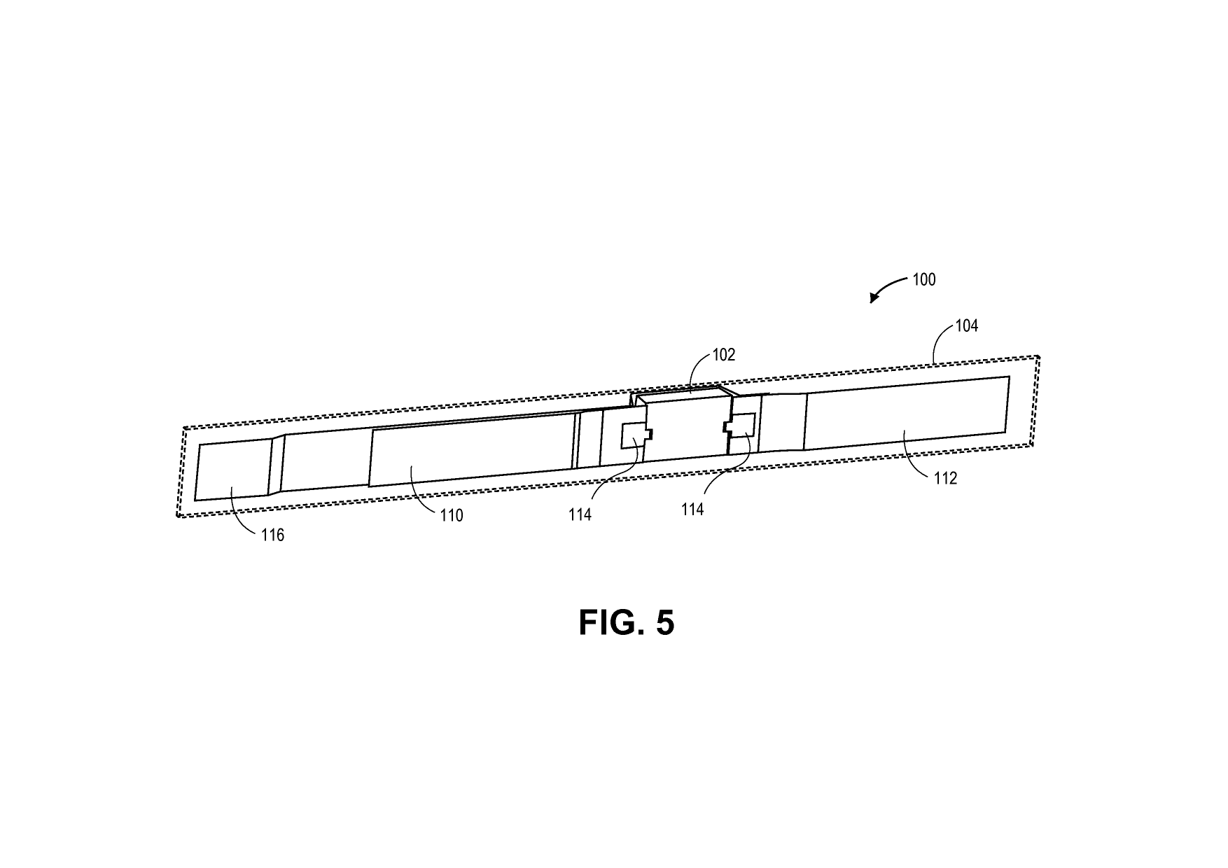

As illustrated in FIG. 5, the electrostatic discharge protective layer 116 may also make direct contact with the skin of a user to provide a grounding path to relieve any accumulation of electrostatic discharge (ESD) and extend over the signal electrode 110. The electrostatic discharge protective layer 116 is disposed at least partially on (e.g., extend along) the inner surface of the strap 104. In some configurations, the electrostatic discharge protective layer 116 may extend over the heart rate monitor 102. In implementations, the electrostatic discharge protective layer 116 may carry a ground signal and substantially cover the signal electrode 110 without being electrically coupled with any element in the strap 104. For instance, the electrostatic discharge protective layer 116 may extend over the heart rate monitor 102 and be mechanically fastened (e.g., sewn in, application of an adhesive, combinations thereof, and so forth) to the ground electrode 112, the heart rate monitor 102, or other portion of the strap 104. In other implementations, the electrostatic discharge protective layer 116 may substantially cover the signal electrode 110 and electrically couple with the ground electrode 112, a connector of the heart rate monitor 102, or a conductive element in the strap 104.

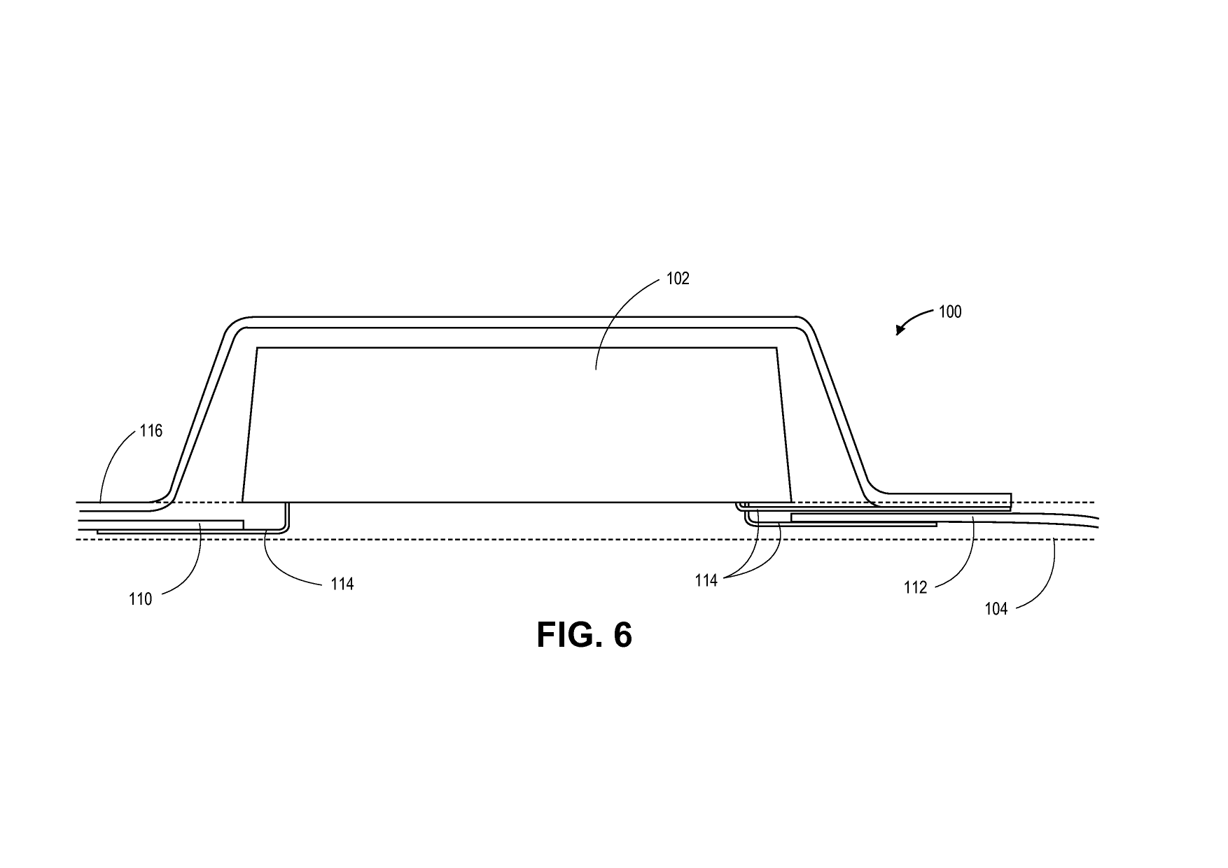

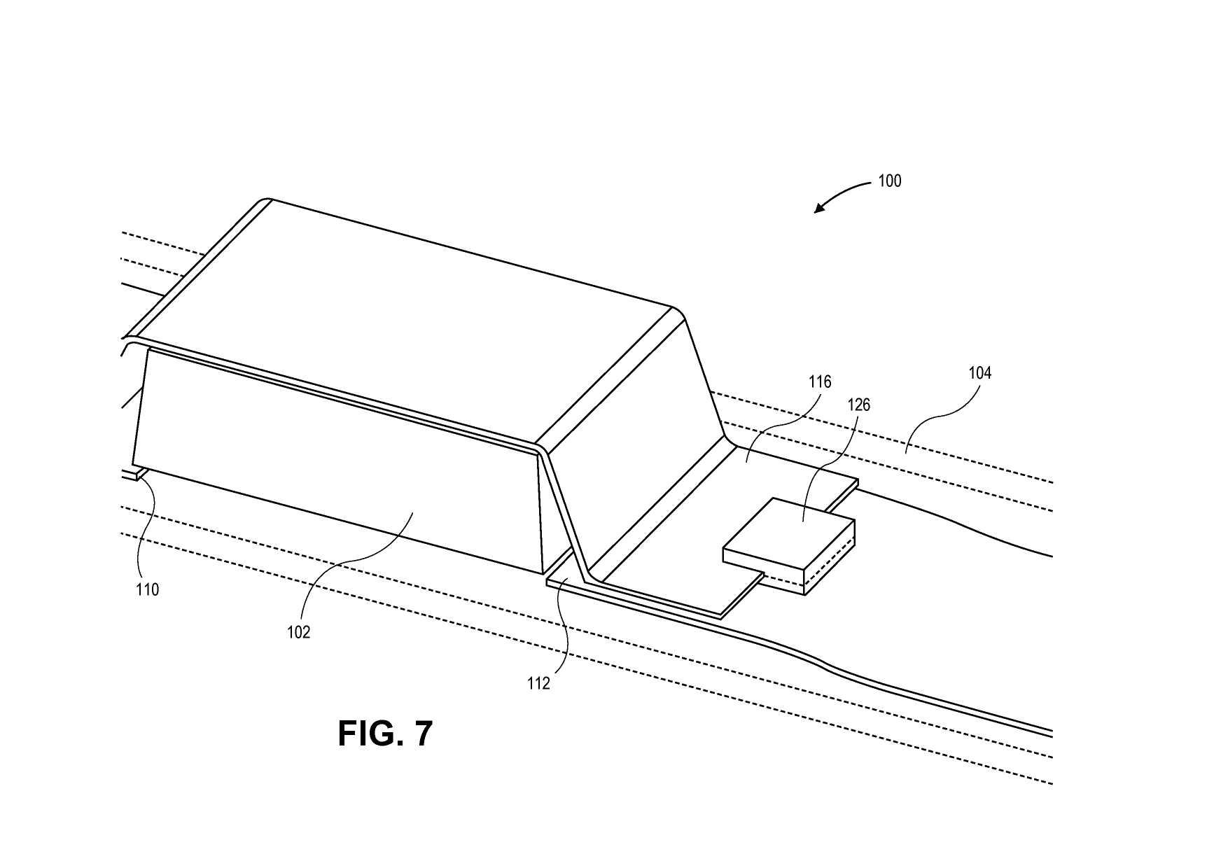

As illustrated in FIG. 6, the electrostatic discharge protective layer 116 may be electrically coupled with a connector 114 of the heart rate monitor. For instance, the electrostatic discharge protective layer may substantially cover the signal electrode 110 and electrically couple with the connector 114 of the heart rate monitor. In some implementations, the electrostatic discharge protective layer 116 may extend over the heart rate monitor 102 so that the heart rate monitor 102 is at least partially positioned between the torso of the user and the electrostatic discharge protective layer 116 when the strap 104 is worn on the body (e.g., about the torso). In other implementations, the electrostatic discharge protective layer 116 may extend under the heart rate monitor 102 so that the electrostatic discharge protective layer 116 is at least substantially positioned between the heart rate monitor 102 and the inner surface of the strap 104.

As illustrated in FIG. 7, the electrostatic discharge protective layer 116 may be electrically coupled to the ground electrode 112 using a coupling element, such as separable connector 126. Electrical coupling may be direct or indirect. In some implementations, the electrostatic discharge protective layer 116 may be electrically coupled with the ground electrode 112 indirectly through a connector that electrically couples the ground electrode 112 to the heart rate module 102. In embodiments, electrical coupling of the electrostatic discharge protective layer 116 and the ground electrode 112 may occur through the user's body or through an intermediate element. Thus, the electrostatic discharge protective layer 116 may be electrically coupled with the ground electrode 112 permanently or separably in any suitable way.

In embodiments, the electrostatic discharge protective layer 116 may extend to cover a substantial portion of signal electrode 110 and extend over the ends and sides of the heart rate module 102 to further shield the heart rate module 102. In other embodiments, the electrostatic discharge protective layer 116 may extend to cover a substantial portion of signal electrode 110 and extend beneath the heart rate module 102.

One or more heart rate monitors 100 can be included as part of a monitoring system (e.g., a heart rate monitoring system 200). With reference to FIG. 1, a heart rate monitoring system 200 may include a heart rate monitor 100 and a device 202, such as a device configured as a watch to be worn around the wrist of a user. In implementations, the device 202 may be a paired device (e.g., a device 202 communicatively paired with the heart rate monitor 102). However, it is contemplated that the device 202 may be any device that is capable of receiving heart rate data transmitted by the heart rate monitor 100. The heart rate monitor 100 can be configured to interpret and/or transmit data describing the heart rate of a user (i.e., information pertaining to the detected electrical impulses and associated with a user's heart rate) to the device 202. The heart rate data can be transmitted wirelessly between the heart rate monitor 100 and the device 202. For example, heart rate data can be transmitted by the heart rate monitor 100 using a radio transmitter, such as a radio frequency (RF) transmitter that transmits a signal containing data describing the measured heart rate to a receiver in the device 202. The transmitted signal may comprise, but is not necessarily limited to: a radio pulse, a coded signal (e.g., Bluetooth, ZigBee, a wireless personal network protocol, such as ANT), and so forth. In implementations, heart rate data can be transmitted in various formats, including, but not necessarily limited to: a pulse signal format, an electrocardiogram (EKG) signal format, a calculated heart rate format, and so forth. However, these formats are provided by way of example only and are not meant to be restrictive of the present disclosure. Thus, other formats may also be used to transmit information associated with or representative of a user's heart rate.

The heart rate monitor 100 may include other sensors such as accelerometers, gyroscopes, magnetic sensors, global positioning system (GPS) receivers, strain gauges, and so forth. The heart rate monitor 100 may utilize information from these sensors to calculate other metrics of interest to the user, including, but not limited to, metrics related to gait efficiency, injury prevention, gait type, torso position, torso orientation relative to ground, and so forth. The heart rate monitor 100 may include internal data storage (e.g., memory), for storing information (data) related to user's heart signals or other metrics. The stored information (data) may be later retrieved by the user for analysis. Storage of information related to heart signals and other metrics is particularly useful in system configurations where the heart rate module 100 is not in communication with other devices, such as device 202, during use (exercise).

The device 202 can include input/output (I/O) instrumentation, such as a display 204, one or more buttons 206 for receiving user input, and so forth. The display 204 can be configured to display the user's heart rate in real time. Further, the device 202 can be configured to provide other monitoring functionality, including, but not necessarily limited to: a current position (e.g., using satellite navigation information, such as Global Positioning System (GPS) information), time, distance, pace, calories burned, and so forth. The device 202 can include a memory for storing information for review and analysis. For example, a memory of the device 202 can be configured to store heart rate data while a user exercises. This data can then be retrieved for review and analysis after the exercise has been completed. Further, the device 202 can include a processor configured to execute programming for providing real time analysis of information, such as information pertaining to a user's heart rate. For example, the device 202 can be configured to use heart rate data to calculate one or more parameters relating to a user's health or fitness, including, but not necessarily limited to: average heart rate during an exercise period, time in a specific heart rate zone, calories burned, breathing rate, heart rate variability, and so forth.

### CONCLUSION

Although the heart rate monitor 100 has been described with reference to example implementations illustrated in the attached drawing figures, it is noted that equivalents may be employed and substitutions made herein without departing from the scope of the invention as recited in the claims. Further, the heart rate monitor 100 and its components as illustrated and described herein are merely examples of a system and components that may be used to implement the present invention and may be replaced with other devices and components without departing from the scope of the present invention.

What is claimed is:

1. A heart rate monitor (HRM), comprising:
a strap configured to be worn on a body of a user, the strap having an inner surface;
a signal electrode disposed at least partially on the inner surface of the strap, the signal electrode configured to receive electrical signals from the body of the user;
a ground electrode disposed at least partially on the inner surface of the strap, the ground electrode configured to electrically ground the heart rate monitor (HRM) to the body of the user; and
an electrostatic discharge protective layer disposed on or in the strap so that the signal electrode is at least substantially positioned between the body of the user and the electrostatic discharge protective layer and the ground electrode at least substantially extends from between the body of the user and the electrostatic discharge protective layer when the strap is worn on the body, the electrostatic discharge protective layer electrically coupled to the ground electrode and configured to at least partially shield the signal electrode from electrostatic discharge.

2. The heart rate monitor as recited in claim 1, wherein the electrostatic discharge protective layer comprises an extension of the ground electrode.

3. The heart rate monitor as recited in claim 1, wherein the electrostatic discharge protective layer comprises a conductive layer coupled to the ground electrode, the ground electrode at least substantially extending from the conductive layer.

4. The heart rate monitor as recited in claim 1, further comprising a heart rate module coupled to the strap, the heart rate module configured to at least one of transmit, receive or store data describing the heart rate of the user.

5. The heart rate monitor as recited in claim 4, wherein a portion of the electrostatic discharge protective layer extends over the heart rate module so that the heart rate module is at least substantially positioned between the body of the user and the electrostatic discharge protective layer when the strap is worn on the body.

6. The heart rate monitor as recited in claim 5, further comprising a sheath at least substantially enclosing the heart rate module and the portion of the electrostatic discharge protective layer extending over the heart rate module.

7. The heart rate monitor as recited in claim 4, further comprising a second electrostatic discharge protective layer electrically coupled to at least one of the electrostatic discharge protective layer or the ground electrode, wherein the second electrostatic discharge protective layer extends over the heart rate module so that the heart rate module is at least partially shielded from electrostatic discharge.

8. The heart rate monitor as recited in claim 7, further comprising a sheath at least substantially enclosing the heart rate module and the second electrostatic discharge protective layer.

9. The heart rate monitor as recited in claim 4, further comprising contacts configured to electrically couple the heart rate module to the signal electrode and the ground electrode, wherein the electrostatic discharge protective layer extends under the heart rate module so that the electrostatic discharge protective layer is at least substantially positioned between the heart rate module and the inner surface of the strap.

10. The heart rate monitor as recited in claim 1, further comprising a separable electrical connector, wherein the electrostatic discharge protective layer is electrically coupled to the ground electrode through the electrical connector.

11. The heart rate monitor as recited in claim 1, wherein the electrostatic discharge protective layer is electrically coupled to the ground electrode through the body of the user.

12. A heart rate monitor (HRM), comprising:
a heart rate module configured to transmit data describing electrical signals from the body of a user to a device;
a strap configured to be worn about the torso of a user to hold the heart rate module proximate to the chest of the user, the strap having an inner surface and an outer surface;
a signal electrode at least partially disposed on the inner surface of the strap and electrically coupled to the heart rate module, the signal electrode configured to receive electrical signals from the heart of the user;
a ground electrode at least partially disposed on the inner surface of the strap and electrically coupled to the heart rate module, the ground electrode configured to electrically ground the heart rate module to the body of the user; and
an electrostatic discharge protective layer at least substantially disposed between the signal electrode and the outer surface of the strap so that the signal electrode is at least substantially covered by the electrostatic discharge protective layer and the ground electrode is at least substantially uncovered by the electrostatic discharge protective layer, the electrostatic discharge protective layer electrically coupled to the ground electrode and configured to at least partially shield the signal electrode from electrostatic discharge.

13. The heart rate monitor as recited in claim 12, wherein the electrostatic discharge protective layer comprises an extension of the ground electrode.

14. The heart rate monitor as recited in claim 12, wherein the electrostatic discharge protective layer comprises a conductive layer electrically coupled to the ground electrode, the ground electrode at least substantially extending from the conductive layer.

15. The heart rate monitor as recited in claim 12, wherein a portion of the electrostatic discharge protective layer extends over the heart rate module so that the heart rate module is at least substantially positioned between the torso of the user and the electrostatic discharge protective layer when the strap is worn about the torso.

16. The heart rate monitor as recited in claim 15, further comprising a sheath at least substantially enclosing the heart rate module and the portion of the electrostatic discharge protective layer extending over the heart rate module.

17. The heart rate monitor as recited in claim 15, further comprising a second electrostatic discharge protective layer electrically coupled to at least one of the electrostatic discharge protective layer or the ground electrode, wherein the second electrostatic discharge protective layer extends over the heart rate module so that the heart rate module is at least partially shielded from electrostatic discharge.

18. The heart rate monitor as recited in claim 17, further comprising a sheath at least substantially enclosing the heart rate module and the second electrostatic discharge protective layer.

19. The heart rate monitor as recited in claim 15, further comprising contacts configured to electrically couple the heart rate module to the signal electrode and the ground electrode, wherein the electrostatic discharge protective layer extends under the heart rate module so that the electrostatic discharge protective layer is at least substantially positioned between the heart rate module and the inner surface of the strap, and wherein the electrostatic discharge protective layer is provided with one or more apertures formed therein through which the contacts extend.

20. The heart rate monitor as recited in claim 12, further comprising a separable electrical connector, wherein the electrostatic discharge protective layer is electrically coupled to the ground electrode through the electrical connector.

21. The heart rate monitor as recited in claim 12, wherein the electrostatic discharge protective layer is electrically coupled to the ground electrode through the body of the user.

22. A heart rate monitoring system, comprising:
a heart rate monitor, the heart rate monitor including a heart rate module configured to transmit a signal containing data describing heart signals of a user; a strap configured to be worn about the torso of the user to hold the heart rate module proximate to the chest of the user; a signal electrode disposed on the inner surface of the strap and electrically coupled to the heart rate module, the signal electrode configured to receive electrical signals from the heart of the user; a ground electrode disposed on the inner surface of the strap and electrically coupled to the heart rate module, the ground electrode configured to electrically ground the heart rate module to the body of the user; and an electrostatic discharge protective layer at least substantially disposed between the signal electrode and the outer surface of the strap so that the signal electrode is at least substantially covered by the electrostatic discharge protective layer and the ground electrode is at least substantially uncovered by the electrostatic discharge protective layer, the electrostatic discharge protective layer electrically coupled to the ground electrode and configured to at least partially shield the signal electrode from electrostatic discharge; and
a device configured to receive the signal containing data describing the heart signals of a user and display information describing the heart signals of the user.

23. The heart rate monitoring system as recited in claim 22, wherein the electrostatic discharge protective layer comprises an extension of the ground electrode.

24. The heart rate monitoring system as recited in claim 22, wherein the electrostatic discharge protective layer comprises a conductive layer coupled to the ground electrode, the ground electrode extending from the conductive layer.
